(19) United States
(12) Patent Application Publication
(10) Pub. No.: US 2014/0271351 A1
(43) Pub. Date: Sep. 18, 2014

(54) WETTING MEDIA OF GLYCEROL AND BUFFER

(71) Applicants: Bo Rud Nielsen, Alleroed (DK); Niels Joergen Madsen, Alleroed (DK); Lars Schoenfeldt, Helsingor (DK)

(72) Inventors: Bo Rud Nielsen, Alleroed (DK); Niels Joergen Madsen, Alleroed (DK); Lars Schoenfeldt, Helsingor (DK)

(73) Assignee: Coloplast A/S, Humlebaek (DK)

(21) Appl. No.: 13/518,192

(22) PCT Filed: Dec. 21, 2010
(86) PCT No.: PCT/DK2010/050353

§ 371 Date: May 2, 2013

(30) Foreign Application Priority Data
Dec. 21, 2009 (DK) PA 2009 70284

(51) Int. Cl. A61L 2/08

(52) U.S. Cl. CPC A61L 2/08; A61L 2/081
USPC 422/22; 604/523

(57) ABSTRACT

Medical device comprising a hydrophilic coating, sterilised while in contact with a swelling medium comprising a low molecular polyol; and a separate buffer selected from the group consisting of carboxylic acids, amino acids, aminosulphonic acids and inorganic acids. The swelling media provides a stable pH after sterilisation and maintain the low friction of the coating.

### FIELD OF THE INVENTION

### Background

It is known to coat medical devices, e.g. catheters for introduction into human cavities, such as blood vessels, digestive organs and the urinary system, with a hydrophilic coating. The coating is as a minimum applied on that part of the surface which is introduced or comes into contact with e.g. mucous membranes during introduction of the device. Whereas such a coating is not particularly slippery when dry, it may become extremely slippery when it is swelled with water before introduction into the human body. The hydrophilic coating thus ensures a substantially painless introduction with a minimum of damage on tissue.

U.S. Pat. No. 3,967,728 to Gordon discloses the use of a sterile lubricant for deposition on and lubricating an uncoated catheter before use.

WO 86/06284 (Astra Meditech Aktiebolag) discloses a wetting and storing device for a coated catheter in which the coating may be wetted using water or water comprising common salt and possibly bactericidal compounds or other additives.

WO 94/16747 discloses a hydrophilic coating with improved retention of water on a surface, especially a surface of a medical device such as a urethral catheter, prepared by applying to the surface, in one or more process steps, at least one solution of components that will combine to form the hydrophilic coating. During the final step the surface is coated with an osmolality promoting agent, which is dissolved or emulsified in the solution or in the last solution to be applied when forming the hydrophilic coating.

Most prior art coatings are developed for instant swelling immediately before use of the medical device on which the coatings are applied. It has been found, however, that most hydrophilic coatings lose their water retention and that the coefficient of friction increases when the coatings are stored in water for an extended period of time, particularly after sterilisation using irradiation or autoclaving.

It is described in EP 1 131 112 that the water retention can be increased dramatically and the initial coefficient of friction can be kept low by carrying out sterilisation of a medical device having a hydrophilic coating while in contact with an aqueous solution comprising hydrophilic polymers, for example polyvinylpyrrolidone. Thus, it seems that the hydrophilic polymers protect the above-mentioned properties during exposure to sterilisation using radiation when wetted with such a polymer solution.

However, there is still a need for methods for providing a sterilised medical device with a hydrophilic coating.

### SUMMARY

The present application discloses that low molecular polyol as part of a swelling media for hydrophilic coated catheters extends the dry-out time for such catheters from a few minutes to more than 10 minutes. However, sterilising hydrophilic coated catheters with water and 1-20% glycerol results in a decrease in pH after sterilisation and storage. The drop in pH can be prevented by adding a buffer to the swelling medium.

### DETAILED DISCLOSURE

One embodiment of the invention relates to a medical device comprising a hydrophilic coating, said medical device being sterilised while in contact with a swelling medium, said swelling medium comprising:

a) a low molecular polyol; and
b) a separate buffer.

A related embodiment relates to a sterilised set comprising a medical device comprising a hydrophilic coating in contact with a swelling medium comprising:

a) a low molecular polyol; and
b) a separate buffer;
wherein said set has been sterilised using irradiation while in contact with said liquid.

The device in this composition can be stored for at least 2 years with retention of the dry-out time and friction-factors important to a medical device.

The medical device may be selected from the group consisting of catheters, endoscopes, laryngoscopes, tubes for feeding, tubes for drainage, guide wires, condoms, urisheaths, barrier coatings, stents and other implants, extra corporeal blood conduits, membranes, blood filters, devices for circulatory assistance, dressings for wound care, and ostomy bags. At present most relevant medical devices or medical device elements are catheters and catheter elements, in particular urinary catheters.

A large number of methods are known for the production of hydrophilic surface coatings for improving the slipperiness of a catheter or other medical device. These methods are most often based on the fact that the substrate to be provided with a hydrophilic surface coating, in the course of one or more process stages with intermediary drying and curing, is coated with one or more (most often two) layers, which are brought to react with one another in various ways, e.g. by polymerisation initiated by irradiation, by UV light, by graft polymerisation, by the formation of inter-polymeric network structures, or by direct chemical reaction. Known hydrophilic coatings and processes for the application thereof are e.g. disclosed in Danish Patent No. 159,018, published European Patent Application Nos. EP 0 389 632, EP 0 379 156, and EP 0 454 293, European Patent No. EP 0 093 093 B2, British Patent No. 1,600,963, U.S. Pat. Nos. 4,119,094, 4,373,009, 4,792,914, 5,041,100 and 5,120,816, and into PCT Publication Nos. WO 90/05162 and WO 91/19756.

In a preferred embodiment the hydrophilic coating is a PVP coating. Such a coating contains PVP bound to the medical device.

In one aspect of the invention the sterilisation by irradiation is performed by β- or γ-irradiation (beta- or gamma-irradiation).

The swelling medium will preferably comprise two important components: the low molecular polyol and the buffer.

The low molecular polyol is preferably selected from the list of glycerol and low-molecular glycols, preferably with molecular weight lower than 200 g/mol, such as ethylene glycol, diethylene glycol, triethylenglycol, propylene glycol, dipropylene glycol, 1,3-propanediol, 1,4-butanediol, and 1,5-pentanediol. These hygroscopic, non-volatile compounds contain 2-3 hydroxyl groups that can hydrogen bond strongly with the polar PVP in the coating and hence plasticize and keep the coating slippery, even if the water in the coating gradually evaporates.

It is preferred to have the low molecular polyol in a concentration of 1 to 5%, more preferred 1-3%.

One aspect of the present invention is contrary to the common belief that a hydrophilic polymer (which has significantly higher molecular weight than 200 g/mol) in the swelling medium is needed to protect the coating during sterilisation and subsequent storage in water. The present data teach that such polymer is not needed. Thus, one aspect of the present invention relates to a swelling medium without a hydrophilic polymer. That is, that the swelling medium comprises less than 3° A), such as less than 2%, or even less than 1% of hydrophilic polymer.

Some polymer might get released from the coating to the swelling medium during storage. However, this is not sufficient to protect the coating during β- or γ-sterilisation. Thus, in a preferred embodiment, the amount of hydrophilic polymer is determined at the time of sealing the package, prior to release from the coating.

In one embodiment, the buffer is a non-polymeric buffer.

In one aspect of the invention, the swelling medium does not contain a hydrophilic polymer without buffer capacity. That is, the swelling medium does not comprise a hydrophilic polymer selected from the group consisting of poly(meth)acrylic acid esters; poly(meth)acrylamides with or without N-alkyl substitution; poly(vinyl alcohol); partially saponified poly(vinyl acetate); poly(ethylene glycol); poly(ethylene glycol-co-propylene glycol); poly(ethylene glycol)-poly(propylene glycol) block copolymers; copolymers and block copolymers of ethylene glycol and other 1,2-epoxide monomers, such as 1-butene oxide, cis- and trans-2-butene oxide, cyclopentene oxide, cyclohexene oxide, and styrene oxide; poly(vinyl methyl ether); poly(2-ethyl-4,5-dihydrooxazole) (e.g. available in various molecular weights as Aquazol from ISP Corporation) and other 2-substituted poly(4,5-dihydrooxazole)s; poly(2-vinyl-1-(3-sulfopropyl)pyridinium inner salt); poly(2-vinyl-1-(4-sulfobutyl)pyridinium inner salt); poly(2-methyl-5-vinyl-1-(3-sulfopropyl)pyridinium inner salt); poly(4-vinyl-1-(3-sulfopropyl)pyridinium inner salt); poly(4-vinyl-1-(4-sulfobutyl)pyridinium inner salt); poly(N,N-dimethyl-N-2-methacryloyloxyethyl-N-(3-sulfopropyl)ammonium inner salt); poly(N,N-dimethyl-N-3-methacrylamidopropyl-N-(3-sulfopropyl)ammonium inner salt); poly(N,N-diethyl-N-methacryloyloxyethoxyethyl-N-(3-sulfopropyl)ammonium inner salt); poly(4-vinyl-N-methylpyridinium-co-p-styrenesulfonate); poly(N,N,N-trimethyl-N-3-methacrylamidopropylammonium-co-2-acrylamido-2-methylpropanesulfonate); poly(methacryloyloxyethyltrimethylammonium-co-2-methacryloyloxyethanesulfonate); poly(N-oxide)s, such as poly(2-vinylpyridine-N-oxide) and poly(4-vinylpyridine-N-oxide); poly(vinylsulfonic acid) and salts; poly(styrenesulfonic acid) and salts; poly(2-methacryloyloxyethanesulfonic acid) and salts; poly(3-methacryloyloxy-2-hydroxypropanesulfonic acid) and salts; poly(2-acrylamido-2-methylpropanesulfonic acid) and salts; poly(3-vinyloxypropanesulfonic acid) and salts; salts of polycarbamoyl sulfonates; salts of sulfonated ethylene-propylene-diene terpolymers; poly(4-vinylbenzyltrimethylammonium salt with a mono- or divalent anion); poly(diallyldimethylammonium salt with a mono- or divalent anion); poly(diallyldiethylammonium salt with a mono- or divalent anion); poly(methacryloyloxyethyltrimethylammonium salt with a mono- or divalent anion); poly(methacryloyloxyethyltriethylammonium salt with a mono- or divalent anion); poly(methacryloyloxypropyltrimethylammonium salt with a mono- or divalent anion); poly(methacryloyloxypropyltriethylammonium salt with a mono- or divalent anion); poly(N-alkyl-2-vinylpyridinium salt with a mono- or divalent anion); poly(N-alkyl-4-vinylpyridine salt with a mono- or divalent anion); and polyurethane ionomers containing tetraalkylammonium groups with mono- or divalent anionic counterions, as described in Encyclopedia of Polymer Science and Engineering, eds. H. F. Mark, N. M. Bikales, C. G. Overberger, and G. Menges, 2. ed., vol. 13, pp. 292-4, Wiley-Interscience, New York, 1988. The cations used for the salts, and the mono- or divalent anions should have no pKₐ values between 2.5 and 8.9, so that they do not affect the buffer capacity of the buffer component between pH 4.0 and 7.4. Appropriate cations for the salts include tetraalkylammonium, trialkylammonium, dialkylammonium, monoalkykammonium, ammonium, alkali metals (i.e. lithium, sodium, potassium, etc.), alkaline earth metals (i.e. magnesium, calcium, etc.), and some trivalent metals (i.e. scandium, yttrium, lanthanum, etc.). Appropriate monovalent anions include chloride, bromide, iodide, nitrate, perchlorate, chlorate, bromate, iodate, chlorite, thiocyanate, hydrogen sulfate, methanesulfonate, trifluoromethanesulfonate, benzenesulfonate, and p-toluenesulfonate. Appropriate divalent anions include sulfate, thiosulfate, and carbonate.

In a preferred embodiment, the swelling medium does not comprise a hydrophilic polymer selected from the group of polysaccharides without carboxylic acid groups (possibly partially hydrolyzed in order to improve solubility and avoid gelation during β-sterilisation), such as agarose; τ-, κ-, λ-, μ-, and ν-carrageenan, and furcellaran; guaran gum; locust bean gum; tamarind flour; scleroglucan; schizophyllan; pseudonigeran; nigeran; isolichenan; amylose; amylopectin; starch and alkylated derivatives, such as hydroxyethylstarch; glycogen; pullulan; dextran; callose; curdlan; pachyman; laminaran; lichenan; cellulose and alkylated derivatives, such as hydroxyethylcellulose or hydroxyproylcellulose; pustulan; alkylated derivatives of chitin, such as hydroxyethylchitin; inulin; levan; α-L-arabinofuranans (e.g. xylopyranoarabinofuranans); β-D-galactans (e.g. arabinogalactans, for example from Larix species); α-D-mannans (e.g. xylomannans; arabinoxylomannans; rhamnomannans; glucomannans; galactofuranomannans); β-D-mannans (e.g. galactomannans); and β-D-xylans (e.g. rhodymenan and arabinoxylans).

In relation to bioburden, the pH of the swelling medium ideally should be as low as possible, but a pH value of about 4 from the time of production to the time of sterilisation works very well. The buffer capacity (and hence the buffer concentration) should be kept as low as possible, because high buffer capacity correlates with the level of pain in small wounds, and the same situation probably applies to catheter users with small scratches in their urethra. Hence, a suitable compromise has been found between conflicting demands for high coating stability (pH>3.7), low bioburden (pH as low as possible, but a pH value of 4 works well), and low buffer capacity (below 4 mM from pH 4 to pH 7.4).

Suitable separate, preferably non-polymeric buffers for addition to low molecular polyols should have at least one suitable acid strength constant, Kₐ, with a pKₐ value between 2 and 6, such as between 2.5 and 5.5, and more preferred between 2.7 and 5. Kₐ and pKₐ are defined for the acid-base equilibrium HA⇄H⁺+A⁻ in water as follows:

Kₐ=[H⁺]×[A⁻]/[HA]; pKₐ=−log₁₀(Kₐ)

The minimum pKₐ value of 2.7 ensures a reasonable buffer capacity at pH 3.7, which is the minimum allowable pH during sterilisation and subsequent storage. Conversely, the maximum pKₐ value of 5.0 ensures a reasonable buffer capacity at the preferred starting pH of 4.0. Buffers that fulfil these requirements include monocarboxylic acids, such as formic acid (pKₐ=3.75), acetic acid (4.75), propionic acid (4.87), 3-hydroxypropionic acid (3.73), 2,3-dihydroxypropionic acid (3.64), gluconic acid (3.56), benzoic acid (4.19), cis-cinnamic acid (3.89), trans-cinnamic acid (4.44), lactic acid (3.85), mandelic acid (3.85), glycolic acid (3.83), phenylacetic acid (4.28), o-chlorobenzoic acid (2.92), m-chlorobenzoic acid (3.82), p-chlorobenzoic acid (3.98), 1-naphthoic acid (3.70), 2-naphthoic acid (4.17), o-toluic acid (3.91), m-toluic acid (4.27), p-toluic acid (4.36), N-acetylglycine (3.67), and hippuric acid (3.80); dicarboxylic acids, such as oxalic acid (pKₐ₁=1.23, pKₐ₂=4.19), malonic acid (pKₐ₁=2.83, pKₐ₂=5.69), succinic acid (pKₐ₁=4.16, pKₐ₂=5.61), glutaric acid (pKₐ₁=4.31, pKₐ₂=5.41), adipic acid (pKₐ₁=4.43, pKₐ₂=5.41), pimelic acid (pKₐ₁=pKₐ₂=4.71), phthalic acid (pKₐ₁=2.89, pKₐ₂=5.51), isophthalic acid (pKₐ₁=3.54, pKₐ₂=4.60), terephthalic acid (pKₐ₁=3.51, pKₐ₂=4.82), 1,1-cyclohexanedicarboxylic acid (pKₐ₁=3.45, pKₐ₂=6.11), malic acid (pKₐ₁=3.40, pKₐ₂=5.11), α-tartaric acid (pKₐ₁=2.98, pKₐ₂=4.34), meso-tartaric acid (pkₐ₁, =3.22, pKₐ₂=4.82), itaconic acid (pKₐ₁=3.85, pKₐ₂=5.45), and fumaric acid (pKₐ₁=3.03, pKₐ₂=4.44); tri- and tetracarboxylic acids, such as citric acid (pKₐ₁=3.14, pKₐ₂=4.77, pKₐ₃=6.39) and 1,2,3,4-butanetetracarboxylic acid (pKₐ₁=3.36, pKₐ₂=4.38, pKₐ₃=5.45, pKₐ₄=6.63); amino acids, such as tryptophan (pKₐ₁=2.83, pKₐ₂=9.39), aspartic acid (pKₐ₁=1.88, pKₐ₂=3.65, pKₐ₃=9.60), glutamic acid (pKₐ₁=2.19, pKₐ₂=4.25, pKₐ₃=9.67), anthranilic acid (o-aminobenzoic acid; pKₐ₁=2.11, pKₐ₂=4.95), m-aminobenzoic acid (4.78), p-aminobenzoic acid (pKₐ₁=2.50, pKₐ₂=4.87), glutathione (3.59), glycylglycine (3.14), glycylglycylglycine (pKₐ₁=3.23, pKₐ₂=8.09), N-phenylglycine (pKₐ₁=1.83, pKₐ₂=4.39), carnosine β-alanylhistidine; pKₐ₁=2.73, pKₐ₂=6.87, pKₐ₃=9.73), niacin (3-pyridinecarboxylic acid; 4.85), 4-pyridinecarboxylic acid (4.96); aminosulphonic acids, such as m-aminobenzenesulfonic acid (3.73), and sulfanilic acid (p-aminobenzenesulfonic acid; 3.23); and inorganic acids, such as hydrofluoric acid (3.45), cyanic acid (3.92), and nitrous acid (3.37). Most pKₐ values are from various editions of The CRC Handbook of Chemistry and Physics, published by The Chemical Rubber Company.

The preferred separate buffers have:
1. As high buffer capacity as possible between the starting pH of 4.0 and the minimum allowable pH of 3.7 in order to prevent pH from falling in this range during β-sterilisation and subsequent storage.
2. As low buffer capacity as possible between pH 4.0 and pH 7.4 in order to minimise the pain felt by users with a damaged urethra.

Hence especially preferred buffers include compounds with only one buffer active group with a pKₐ value between 3.7 and 4.0 such as the monocarboxylic acids, formic acid, cis-cinnamic acid, lactic acid, 3-hydroxypropionic acid, mandelic acid, glycolic acid, 1-naphthoic acid, o-toluic acid, m-chlorobenzoic acid, p-chlorobenzoic acid, N-acetylglycine, hippuric acid, m-aminobenzenesulfonic acid, and the inorganic cyanic acid. Especially preferred buffers with several buffer active groups (such as di-, tri- or polyacids, or amino acids) include compounds with one or several pKₐ values between 3.7 and 4.0 and the other pKa values smaller than 3.7 or larger than 8.9 (so the buffer capacity between 4.0 and 7.4 is negligible), such as aspartic acid and glutathione.

Buffers with the largest pKₐ value below 3.7 are slightly less preferred because of their rather low buffer capacity at pH 4.0. However, if very low buffer capacity between pH 4.0 and 7.4 is of paramount importance, then buffers with the largest pKₐ value below 3.7 are ideal; these include 2,3-dihydroxypropionic acid, gluconic acid, o-chlorobenzoic acid, glycylglycine, sulfanilic acid, hydrofluoric acid, and nitrous acid. Slightly less preferred buffers with several buffer active groups include compounds with one or several pKₐ values below 3.7 and the other pKa values larger than 8.9, such as tryptophan.

Buffers with one or several pKₐ values between 4.0 and 8.9 are less preferred, because their buffer capacities between 4.0 and 8.9 do not contribute very much to the stabilisation of pH between 3.7 and 4.0 and, at the same time, may contribute significantly to the pain felt by the user. However, it is still better to employ one of these buffers than none at all; they include acetic acid, propionic acid, benzoic acid, trans-cinnamic acid, phenylacetic acid, 2-naphthoic acid, m-toluic acid, p-toluic acid, oxalic acid, malonic acid, succinic acid, glutaric acid, adipic acid, pimelic acid, phthalic acid, isophthalic acid, terephthalic acid, 1,1-cyclohexanedicarboxylic acid, malic acid, α-tartaric acid, meso-tartaric acid, itaconic acid, fumaric acid, citric acid, 1,2,3,4-butanetetracarboxylic acid, glutamic acid, glycylglycylglycine, anthranilic acid, m-aminobenzoic acid, p-aminobenzoic acid, N-phenylglycine, carnosine, niacin, and 4-pyridinecarboxylic acid.

The buffer capacity, β, of the swelling media was measured as it is standard in the art, see e.g. Niels Linnet: “pH measurements in theory and practice”, 1. ed., Radiometer A/S, Copenhagen, 1970:

β=db/dpH

where db is the amount of strong base (measured in moles) per litre of the swelling medium required to bring about the pH change dpH in the solution. If, for example, 0.13 mL 0.1 M NaOH (=0.013 mmol=13 μmol) was required to raise pH from 7.40 to 7.94 in 20 mL of a certain swelling medium, then the buffer capacity, β, at pH 7.67 (the mean value of 7.40 and 7.94) was:

β(7.67)=db/dpH=(0.65 μmol/mL NaOH)/(7.94−7.40)=1.2 μmol/(pH×mL)=1.2 mM/pH

Hence, the more NaOH that was needed to raise the pH by a certain amount, the higher the buffer capacity. According to theory, the maximum buffer capacity of a buffer active substance is found at pH=pKₐ of the group and is equal to 0.576 times the concentration of the buffer active group.

Buffer capacity data are presented below as the number of micromoles of NaOH required to bring 1 mL swelling medium from pH 4.0 to 7.4. The unit of this buffer capacity is μmol/mL=mmol/L=mM. In some cases the buffer capacity was measured as the number of micromoles of HCl required to bring 1 mL swelling medium from pH 7.4 to 4.0. The titrations with NaOH and HCl should in principle give exactly the same buffer capacity, but in reality the buffer capacity measured from the HCl titration is slightly higher than the buffer capacity from the NaOH titration. This is because the HCl titration moves from high to low pH, that is from an alkaline to an acidic solution, and it is difficult to prevent the alkaline sample from absorbing CO₂ from the air. As noted above, CO₂ will be converted in the alkaline sample to buffer-active CO₃²⁻ or HCO₃⁻, and this will give rise to an artificially high reading of buffer capacity. However, control measurements showed that this was no problem in the present system.

In a preferred embodiment of the invention, the buffer capacity of the separate buffer is below 8, such as below 7, preferably below 6, or even 5, most preferably below 4 mM in the interval from pH 4 to pH 7.4.

### EXAMPLES

### Example 1

### Dry-Out Time Measurements

### Materials

150 dry male SpeediCath CH14 polyurethane catheters with a cross linked, stable PVP coating
Water- and vapor tight package for all catheters

### Glycerol

Distilled water

### Swelling Media

I: 0 g glycerol and 1000 ml Dest H₂O
II: 50 g glycerol and 1000 ml Dest H₂O
III: 100 g glycerol and 1000 ml Dest H₂O
IV: 10 g glycerol and 1000 ml Dest H₂O
V: 30 g glycerol and 1000 ml Dest H₂O

### Procedure

30 One Coat CH14 male catheters were packaged individually with 10 ml of swelling media I-V. They were then sterilised using 2×27.5 kGy electron beam (β) irradiation.

Some catheters were tested immediately, whereas others were stored at 40° C. for 1 or 3 months prior to testing, corresponding to 4 or 12 months at 20° C., respectively (Q₁₀ value of 2).

### Analysis

The dry-out time was found by hanging a number of catheters vertically at time 0 minutes and then subjectively determining the time when the coating turned tacky instead of slippery. The time was determined using a stop watch until a maximum of 10 minutes.

### Results

TABLE 1
Dry-out time for sterilised catheters after 1, 2 and 3 months of storage at
40° C. after sterilisation with various concentrations of
glycerol in the swelling media

| % glycerol | T = 0 | T = 1 mth | T = 2 mths | T = 3 mths |
|---|---|---|---|---|
| 0 | 4-6 | 2-5 | 3-5 | |
| 1 | >10 | 9-10 | >10 | 9-10 |
| 3 | >10 | >10 | >10 | >10 |
| 5 | 9-10 | >10 | >10 | |
| 10 | 9-10 | >10 | >10 | |

The results showed that using just 1% of glycerol in the swelling media dramatically increased the dry-out time. Higher concentrations of glycerol gave the same good results.

### Example 2

### pH Measurements

pH was measured in the samples above. pH before sterilisation was 4.0. A marked decrease in pH was observed after sterilisation. A further decrease in pH was observed after storage.

TABLE 2
pH for sterilised catheters after 1, 2 and 3 months of storage at 40° C. after
sterilisation with various concentrations of glycerol in the swelling media

| % glycerol | T = 0 | T = 1 mth | T = 2 mths | T = 3 mths |
|---|---|---|---|---|
| 0 | 3.73-3.81 | 3.7 | 3.71 | |
| 1 | 3.68 | 3.64 | 3.65 | 3.6 |
| 3 | 3.67 | 3.65 | 3.65 | 3.57 |
| 5 | 3.72 | 3.58 | 3.64 | |
| 10 | 3.77 | 3.58 | 3.63 | |

### Example 3

### pH and Friction Measurements with Glycerol and Buffer in the Swelling Media

### Materials

150 dry male SpeediCath CH12 polyurethane catheters with a cross linked, stable PVP coating
Water- and vapor tight package for all catheters

### Glycerol

### NaCl

Distilled water

### Swelling Media

A: (1% Glycerol) 1000 mL dest. H₂O+9 g NaCl+10 g glycerol+0.23 g Formic acid
B: (3% Glycerol) 1000 mL dest. H₂O+9 g NaCl+30 g glycerol+0.23 g Formic acid
C: (5% Glycerol) 1000 mL dest. H₂O+9 g NaCl+50 g glycerol+0.23 g Formic acid

1N NaoH was used for adjusting the mixtures to pH 4.

One Coat CH12 male catheters were packaged individually with 10 ml of swelling media A-C. They were then sterilised using 2×27.5 kGy electron beam (β) irradiation.

TABLE 3
pH before and after sterilisation with buffer and various concentrations of
glycerol in the swelling media

| | pH before | pH 24 h after | Osmolality 24 h |
|---|---|---|---|
| | sterilisation | sterilisation | after steriliation |
| 1% glycerol | 4.05 | 4.03 | 395 mOsmol/kg |
| 3% glycerol | 3.90 | 3.89 | 599 mOsmol/kg |
| 5% glycerol | 4.04 | 3.99 | 808 mOsmol/kg |

As can be seen from Table 3, the presence of buffer in the swelling medium provides a stable pH after sterilisation. The resulting sterilised catheters still show same good dry-out time and low friction, see Table 4.

TABLE 4
Friction 24 hours after sterilisation for catheters with buffer and various
concentrations of glycerol in the swelling media

| | mN |
|---|---|
| 1% Glycerol | 36 |
| 3% Glycerol | 31 |
| 5% Glycerol | 31 |

1. Medical device comprising a hydrophilic coating, sterilised while in contact with a swelling medium comprising:
a) a low molecular polyol; and
b) a separate buffer selected from the group consisting of carboxylic acids, amino acids, aminosulphonic acids and inorganic acids.

2. Medical device according to claim 1, wherein the medical device is a hydrophilic coated catheter.

3. Medical device according to claim 1, wherein the hydrophilic coating is a PVP coating.

4. Medical device according to claim 1, sterilised using radiation.

5. Medical device according to claim 1, wherein the low molecular polyol has a molecular weight below 200 g/mol.

6. Medical device according to claim 1, wherein the low molecular polyol is present in the swelling medium in a concentration of 0.1% to 20%.

7. Medical device according to claim 1 wherein the low molecular polyol is glycerol.

8. Medical device according to claim 1, wherein the separate buffer is a non-polymeric buffer.

9. Medical device according to claim 1, wherein the separate buffer is a buffer with at least one pKₐ value between 2.7 and 5.

10. Medical device according to claim 1, wherein the buffer capacity is below 4 mM from pH 4 to pH 7.4.

11. A sterilised set comprising a medical device comprising a hydrophilic coating in contact with an aqueous liquid comprising:
a) a low molecular polyol;
b) a separate buffer;
wherein said set has been sterilised using irradiation while in contact with said liquid.

12. Sterilised set according to claim 11, wherein the device is a hydrophilic coated catheter.

13. Sterilised set according to claim 11, wherein the hydrophilic coating contains PVP.

14. Sterilised set according to claim 11, sterilised using β- or γ-irradiation.

15. Sterilised set according to claim 11, wherein the low molecular polyol has a molecular weight below 200 g/mol.

16. Sterilised set according to claim 11, wherein the low molecular polyol is present in the swelling medium in a concentration of 0.1% to 20%.

17. Sterilised set according to claim 11, wherein the separate buffer is a non-polymeric buffer.

18. Sterilised set according to claim 11, wherein the separate buffer is a buffer with at least one pKₐ value between 2.7 and 5.

19. Sterilised set according to claim 11, wherein the buffer capacity is below 4 mM from pH 4 to pH 7.4.

20. A method for sterilising a medical device comprising a hydrophilic coating using radiation, said method comprising the steps of bringing the medical device, having such a coating, in contact with an aqueous liquid for wetting the hydrophilic coating, said liquid comprising a solution of a low molecular polyol and a separate buffer, and sterilising the device by applying a sufficient amount of radiation.

21. The method according to claim 20, wherein the device is a hydrophilic coated catheter.

22. The method according to claim 20, wherein the hydrophilic coating contains PVP.

23. The method according to claim 20, wherein the sterilisation is by β- or γ-irradiation.

24. The method according to claim 20, wherein the low molecular polyol has a molecular weight below 200 g/mol.

25. The method according to claim 20, wherein the low molecular polyol is present in the swelling medium in a concentration of 0.1% to 20%.

26. The method according to claim 20, wherein the separate buffer is a non-polymeric buffer.

27. The method according to claim 20, wherein the separate buffer is a buffer with at least one pKₐ value between 2.7 and 5.

28. The method according to claim 20, wherein the buffer capacity is below 4 mM from pH 4 to pH 7.4.
